(19) United States
(12) Patent Application Publication
(10) Pub. No.: US 2025/0388600 A1
(43) Pub. Date: Dec. 25, 2025

(54) A PROCESS FOR THE PREPARATION OF EDOXABAN AND ITS INTERMEDIATES

(71) Applicant: AMI LIFESCIENCES PRIVATE LIMITED, Vadodara (IN)

(72) Inventors: Kalpesh Ravajibhai Patel, Vadodara (IN); Virendra Haridas Thakrar, Vadodara (IN); Tushar Bharatkumar Mehta, Vadodara (IN); Nitin Vasantbhai PRAJAPATI, Vadodara (IN); Nirav Bavanjibhai SUTARIYA, Vadodara (IN); Kripalsingh Ajitsingh SUNDARANI, Vadodara (IN); Shyam Kishorbhai BHALANI, Vadodara (IN)

(73) Assignee: AMI LIFESCIENCES PRIVATE LIMITED, Vadodara (IN)

(21) Appl. No.: 18/879,299

(22) PCT Filed: Jul. 3, 2023
(86) PCT No.: PCT/IB2023/056894

§ 371 Date: Dec. 27, 2024

(30) Foreign Application Priority Data
Jul. 4, 2022 (IN) 202221038289

(51) Int. Cl. C07D 513/04; C07D 213/75

(52) U.S. Cl. CPC C07D 513/04; C07D 213/75

(57) ABSTRACT

The present invention relates to an efficient and industrially advantageous process for the preparation of Edoxaban of Formula-I or salt thereof. The present invention also relates to a process for preparation of Edoxaban intermediates namely methyl 2-[(5-chloropyridin-2-yl)amino]-2-oxoacetate hydrochloride of Formula-II, tert-Butyl [(1R,2S,5S)-2-[[2-[(5-chloropyridin-2-yl)amino]-2-oxoacetyl]amino]-5-(dimethylaminocarbonyl)cyclohexyl]carbamate of Formula-IV, and their use for the preparation of Edoxaban or salt thereof.

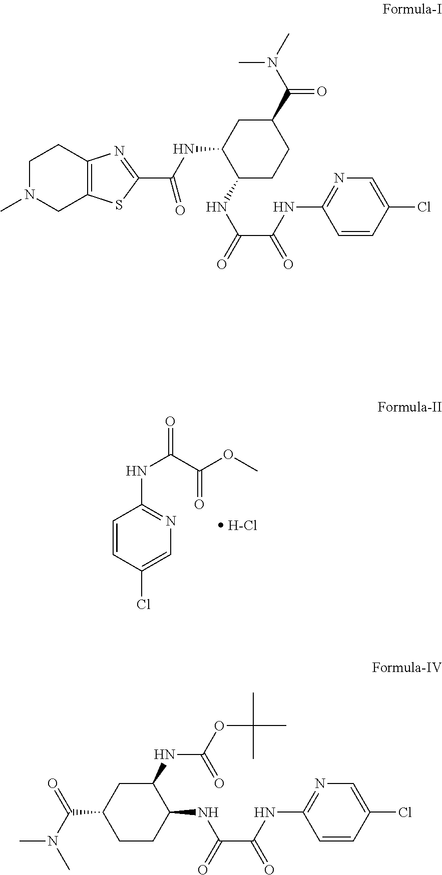

### FIELD OF THE INVENTION

The present invention relates to an efficient and industrially advantageous process for the preparation of Edoxaban or salt thereof.

The present invention also relates to a process for the preparation of Edoxaban intermediates namely methyl 2-[(5-chloropyridin-2-yl)amino]-2-oxoacetate hydrochloride of Formula-II, tert-Butyl [(1R,2S,5S)-2-[[2-[(5-chloropyridin-2-yl)amino]-2-oxoacetyl]amino]-5-(dimethylaminocarbonyl)cyclohexyl]carbamate of Formula-IV, and their use for the preparation of Edoxaban or salt thereof.

### BACKGROUND OF THE INVENTION

Edoxaban tosylate monohydrate is chemically known as N-(5-chloropyridin-2-yl)-N′-[(1S,2R,4S)-4-(N,N-dimethylcarbamoyl)-2-(5-methyl-4,5,6,7-tetrahydro[1,3]thiazolo[5,4-c]pyridine-2-carboxamido)cyclohexyl]oxamide mono(4-methylbenzenesulfonate) monohydrate, having the structure of Formula-A.

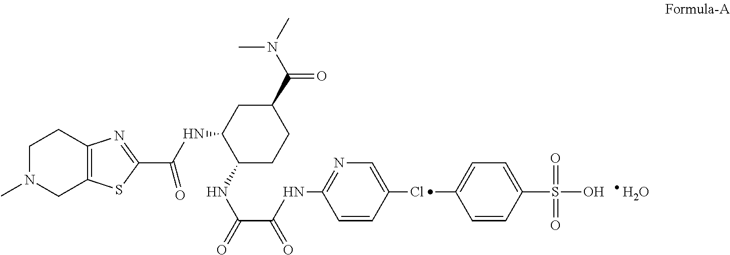

Edoxaban tosylate monohydrate has been developed by Daiichi Sankyo and approved by USFDA on Jan. 8, 2015, under the proprietary name SAVAYSA®. Edoxaban is an oral anticoagulant drug indicated to reduce the risk of stroke and systemic embolism in patients with nonvalvular atrial fibrillation. It is also indicated for the treatment of deep vein thrombosis and pulmonary embolism.

European patent number EP 1405852B1 (herein after EP '852) first discloses Edoxaban tosylate monohydrate. The process to prepare Edoxaban tosylate monohydrate has also been disclosed by reacting compound of Formula-Ia with compound of Formula-Ib in presence of sodium hydrogen carbonate to obtain compound of Formula-IIa. The resulting compound is then reacted with lithium hydroxide in presence of water to obtain compound of Formula-IIb, which on further reaction with compound of Formula-III in presence of 1-hydroxybenzotriazole (HOBT) monohydrate and 1-ethyl-3-(3-dimethylaminopropyl) carbodiimide hydrochloride (EDC·HCl) followed by purification using column chromatography results into the compound of Formula-IV. Resulting compound is then deprotected to obtain compound of Formula-V followed by its reaction with compound of Formula-VIa in presence of condensing agent 1-ethyl-3-(3-dimethylaminopropyl) carbodiimide hydrochloride (EDC·HCl) and 1-hydroxybenzotriazole (HOBT) to obtain Edoxaban compound of Formula-I. Resulting Edoxaban is then converted to its salt.

The schematic representation of above process is depicted as below in scheme-01:

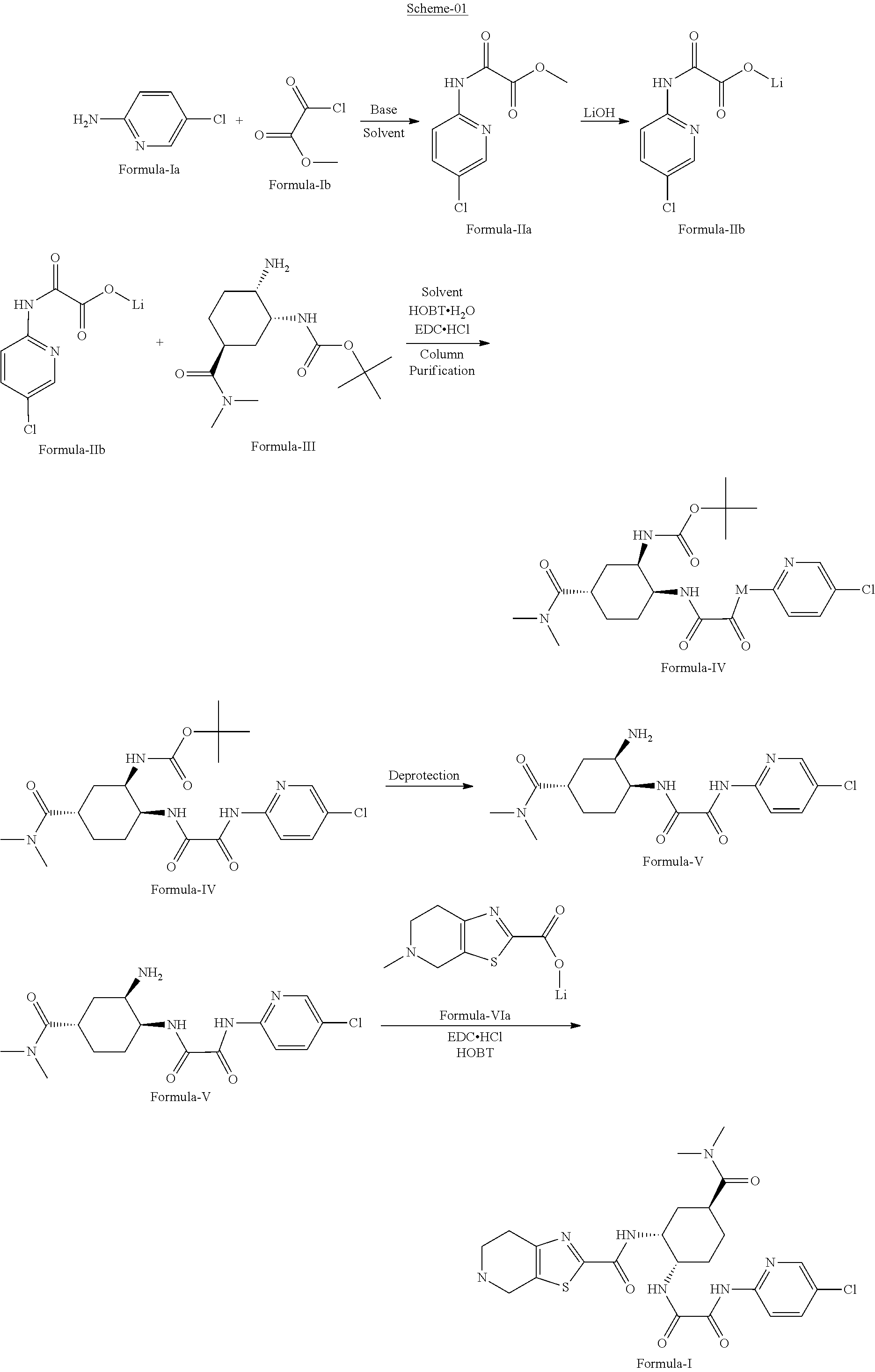

The above process requires use of base for the preparation of compound of the Formula-IIa. Also, the process required diethyl ether and ammonium chloride during the work-up. This process fails to produce pure methyl 2-[(5-chloropyridin-2-yl)amino]-2-oxoacetate hydrochloride of Formula-Ila and requires additional purification method. Further, EP '852 process require catalyst 1-hydroxybenzotriazole (HOBT) in excess amount for the condensation of Lithium salt of Formula-IIb and Formula-III. Also, the condensation reaction require longer reaction time. Generally, HOBT contains hydrazine moiety. When HOBT is used in any reaction, hydrazine moiety present may result into potential genotoxic impurity in product, which may carry forward in final Edoxaban API and may requires additional purification to control the impurities which may affect yield & purity of final Edoxaban API. The process disclosed in EP '852 is not an attractive option to use for industrial scale as it requires longer reaction time, tedious work-up process, and results into lower yield & purity.

US patent application number US20050245565 (herein after US '565) discloses a process for the preparation of compound of Formula-Ila comprising reacting compound of Formula-Ia with compound of Formula-Ic in presence of triethylamine at room temperature for 14 hours. After completion of reaction, aqueous solution of sodium hydrogen carbonate is added to reaction mixture. The resulting mixture is then allowed to settle to separate the organic and aqueous layers. Resulting organic layer is then distilled followed by purification using column chromatography results into compound of Formula-Ila with yield of 22.27%.

The schematic representation of above process is depicted as below in scheme-02:

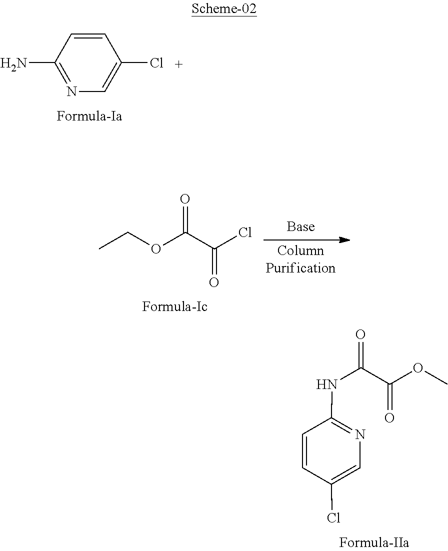

Major drawbacks of this process are use of organic base, longer reaction time, tedious work-up process, column chromatography and lower yield, which makes the process unsuitable for industrial scale.

Indian patent application number IN 201741033706 (herein after IN '706) discloses a process for the preparation of Edoxaban comprising reacting compound of Formula-IIb with compound of Formula-IIIa in presence of N,N-diisopropylethylamine (DIPEA), pyridinium p-toluene sulfonate (PPTS), 1-ethyl-3-(3-dimethylaminopropyl) carbodiimide hydrochloride (EDC·HCl) and 1-hydroxybenzotriazole (HOBT) for 16 hours to 20 hours to obtain reaction mixture. The resulting mixture is treated with sodium bicarbonate and citric acid. The obtained mixture is allowed to separate the layers and organic layer is distilled followed by treatment with methyl tertiary butyl ether to obtain compound of Formula-IV. The resulting compound is then reacted with methane sulfonic acid and triethylamine followed by further reaction with compound of Formula-VI in presence of 1-hydroxybenzotriazole (HOBT) and 1,3-dicyclohexylcarbodiimide (DCC) to obtain reaction mixture. Resulting mixture is then treated with cyclohexane and methanol to obtain Edoxaban compound of Formula-I.

The schematic representation of above process is depicted as below in scheme-03:

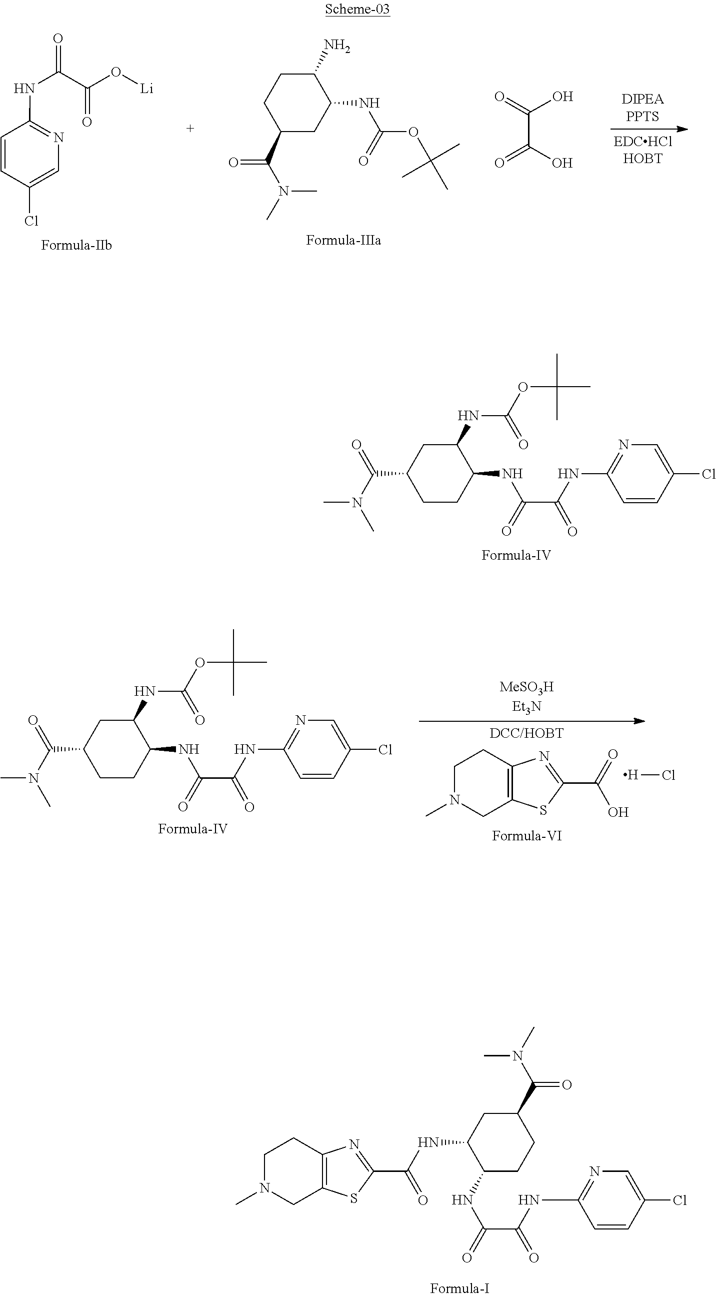

The above process requires longer reaction time, tedious work-up process, lower yield. Also, the obtained purity of compound Formula-IV and Edoxaban compound of Formula-I is not mentioned. Further, during reaction of compound of Formula-IIb with compound of Formula-IIIa and for the conversion of compound of Formula-IV to Edoxaban compound of Formula-I, excess amount of HOBT is used. Generally, HOBT contains hydrazine moiety. When HOBT is used in any reaction, hydrazine moiety present may result into potential genotoxic impurity in product, which may carry forward in final Edoxaban API and may require additional purification to control the impurities and may affect yield & purity of final Edoxaban API. Thus, the process disclosed in IN '706 is not an attractive option to use for industrial scale as it requires longer reaction time, tedious work-up process, and results into lower yield & purity.

PCT publication number WO2018/011823 (herein after WO '823) discloses a process for the preparation of Edoxaban tosylate hydrate of Formula-A comprising deprotecting compound of Formula-IV using methane sulfonic acid and presence of dichloromethane followed by reaction with compound of Formula-VI in presence of triethylamine (TEA), 1-ethyl-3-(3-dimethylaminopropyl) carbodiimide hydrochloride (EDC·HCl) and 1-hydroxybenzotriazole (HOBT) at room temperature to obtain Edoxaban of Formula-I with yield of 85%. The resulting Edoxaban is reacted with p-toluene sulfonic acid (PTSA) in presence of dichloromethane (MDC) and ethanol to obtain Edoxaban tosylate monohydrate of Formula-A with overall yield of 63%.

The schematic representation of above process is depicted as below in scheme-04:

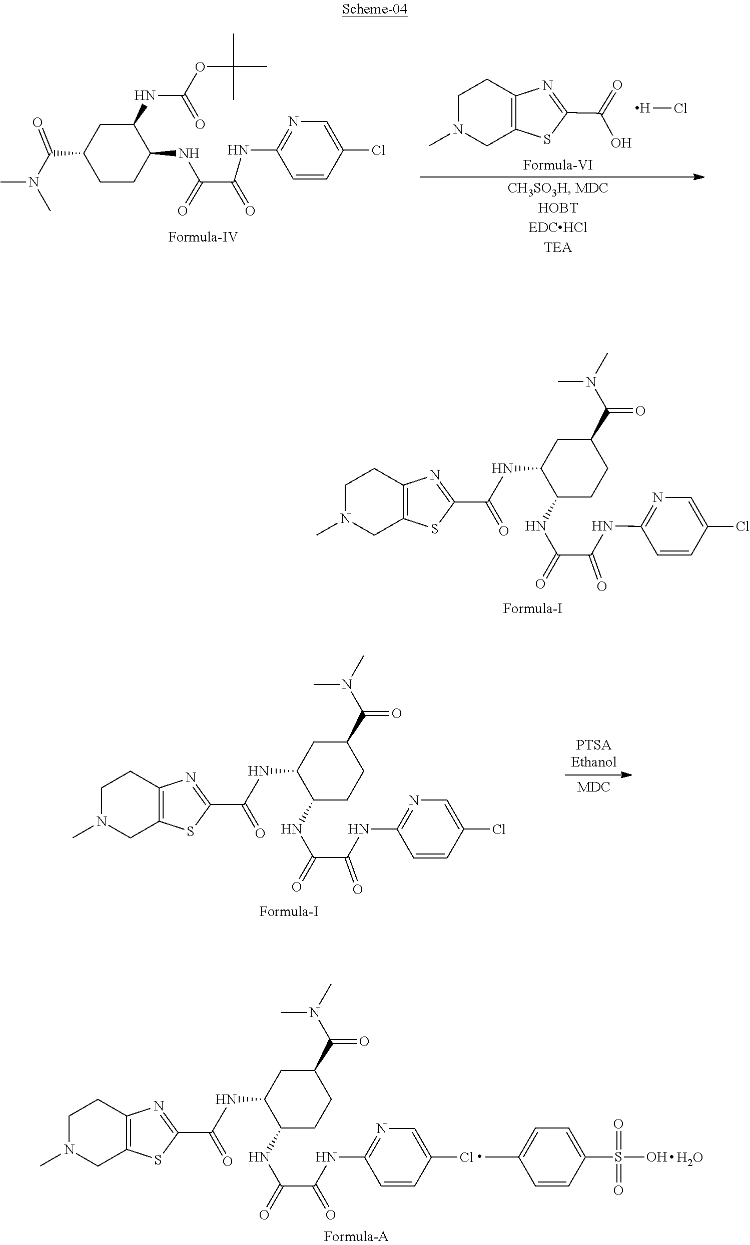

The above process requires HOBT. Generally, HOBT contains hydrazine moiety. When HOBT is used in any reaction, hydrazine moiety present may result into potential genotoxic impurity in product, which may carry forward in final Edoxaban API and may requires additional purification to control the impurities which may affect yield & purity of final Edoxaban API. Thus, process disclosed in WO '823 is not an attractive option to use for industrial scale.

Another PCT publication number WO2021/001728 (herein after WO '728) discloses a process for the preparation of Edoxaban tosylate hydrate of Formula-A comprising reacting compound of Formula-V with compound of Formula-VI in presence of triethylamine, 1-hydroxybenzotriazole (HOBT) & 1-ethyl-3-(3-dimethylaminopropyl) carbodiimide hydrochloride (EDC·HCl) to obtain Edoxaban compound of Formula-I. Resulting Edoxaban is reacted with p-toluenesulfonic acid (PTSA) in presence of isopropanol (IPA) and water to obtain Edoxaban tosylate monohydrate compound of Formula-A with yield of 60%.

The schematic representation of above process is depicted as below in scheme-05:

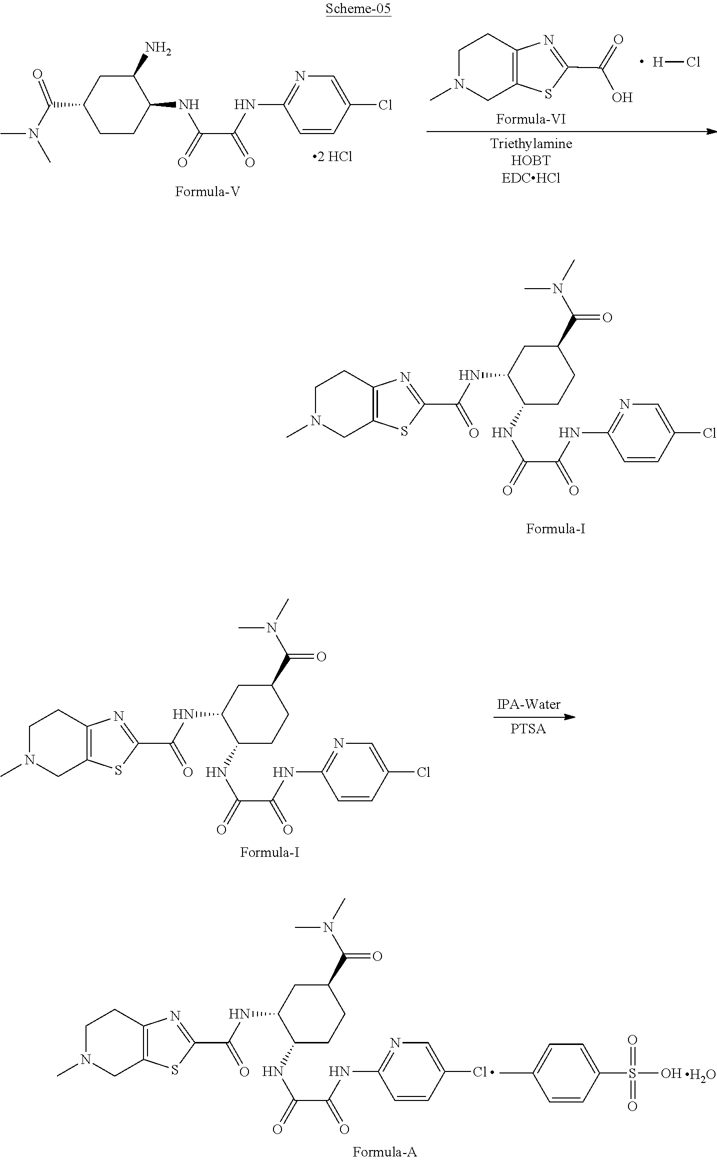

The above process requires longer reaction time, tedious workup process and use of excess amount of HOBT which may results into potential genotoxic impurities. Thus, process disclosed in WO '728 is not an attractive option to use for industrial scale.

Prior art references required tedious work up process, column chromatography, 1-hydroxybenzotriazole (HOBT) which may result into potential genotoxic impurities, longer reaction time, lower yield, or purity. Therefore, there is an urgent need for an improved process for the preparation of Edoxaban compound of Formula-I or salt thereof having high purity and high yield which overcomes the drawbacks of the prior arts process.

The present inventors have developed an efficient process for the preparation of Edoxaban or salt thereof and its intermediates which offer advantage over the prior art processes in terms of high yield, high purity and less effluents and simple scalable procedure suitable for large scale industrial production of Edoxaban compound of Formula-I or salt thereof.

### OBJECTS OF THE INVENTION

The main object of the present invention is to provide an efficient and industrially advantageous process for the preparation of Edoxaban of Formula-I or salt thereof having higher yield and purity.

Another object of the present invention is to provide an industrially advantageous process for the preparation of Edoxaban intermediates namely methyl 2-[(5-chloropyridin-2-yl)amino]-2-oxoacetate hydrochloride of Formula-II, tert-Butyl [(1R,2S,5S)-2-[[2-[(5-chloropyridin-2-yl)amino]-2-oxoacetyl]amino]-5-(dimethylaminocarbonyl)cyclohexyl]carbamate of Formula-IV, and their use for the preparation of Edoxaban or salt thereof.

### SUMMARY OF THE INVENTION

First aspect of the present invention is to provide a process for preparation of Edoxaban of Formula-I or salt thereof,

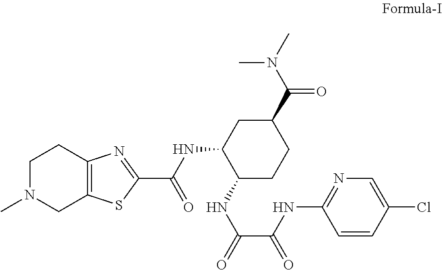 [SMILES: CN1CCc2nc(C(=O)N[C@@H]3C[C@@H](C(=O)N(C)C)CC[C@@H]3NC(=O)C(=O)Nc3ccc(Cl)cn3)sc2C1]

comprising the steps of:
a) reacting compound of Formula-Ia,

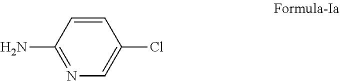

with compound of Formula-Ib,

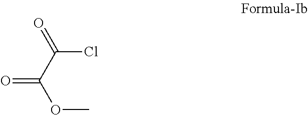

using solvent in absence of base to obtain compound of Formula-II;

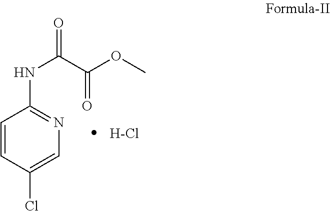

b) reacting compound of Formula-II with compound of Formula-III,

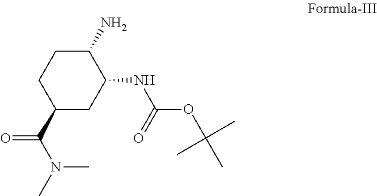 [SMILES: CN(C)C(=O)[C@H]1CC[C@H](N)[C@H](NC(=O)OC(C)(C)C)C1]

in presence of base and solvent to obtain compound of Formula-IV; and

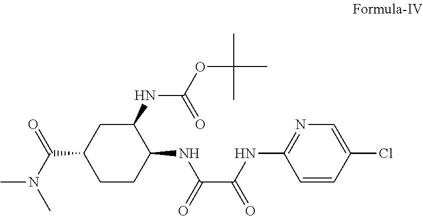 [SMILES: CN(C)C(=O)[C@H]1CC[C@H](NC(=O)C(=O)Nc2ccc(Cl)cn2)[C@H](NC(=O)OC(C)(C)C)C1]

c) converting compound of Formula-IV to Edoxaban of Formula-I or salt thereof.

Second aspect of the present invention is to provide a process for preparation of compound of Formula-II,

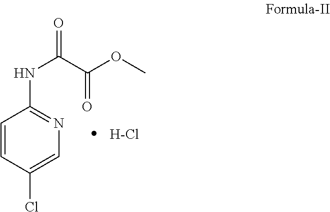

comprising reacting compound of Formula-Ia,

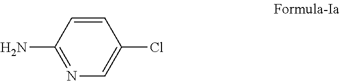

with compound of Formula-Ib,

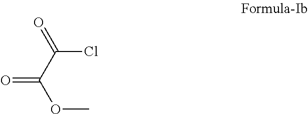

using solvent in absence of base to obtain compound of Formula-II.

Third aspect of the present invention is to provide a process for preparation of compound of Formula-IV,

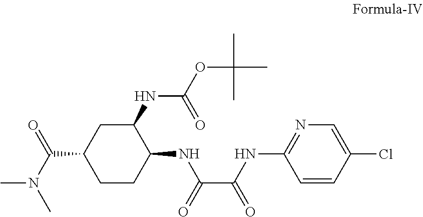 [SMILES: CN(C)C(=O)[C@H]1CC[C@H](NC(=O)C(=O)Nc2ccc(Cl)cn2)[C@H](NC(=O)OC(C)(C)C)C1]

comprising reacting compound of Formula-II,

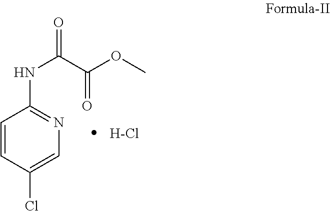

with compound of Formula-III,

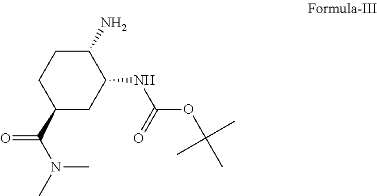 [SMILES: CN(C)C(=O)[C@H]1CC[C@H](N)[C@H](NC(=O)OC(C)(C)C)C1]

in presence of base and solvent to obtain compound of Formula-IV.

Fourth aspect of the present invention is to provide a process for preparation of Edoxaban compound of Formula-I or salt thereof,

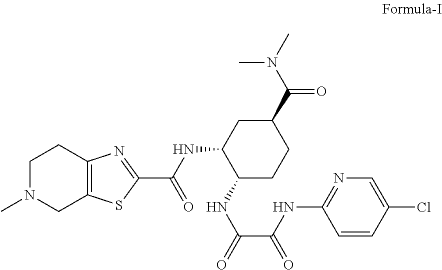 [SMILES: CN1CCc2nc(C(=O)N[C@@H]3C[C@@H](C(=O)N(C)C)CC[C@@H]3NC(=O)C(=O)Nc3ccc(Cl)cn3)sc2C1]

comprising reacting compound of Formula-V,

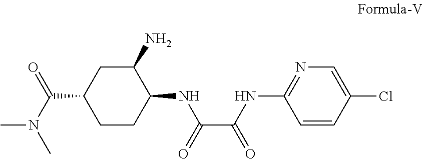 [SMILES: CN(C)C(=O)[C@H]1CC[C@H](NC(=O)C(=O)Nc2ccc(Cl)cn2)[C@H](N)C1]

with compound of Formula-VI,

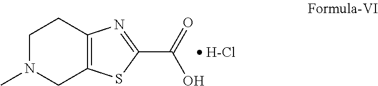

in presence of base, condensing agent, and 2-Hydroxy pyridine-1-oxide (HOPO) as an additive to obtain Edoxaban compound of Formula-I or salt thereof.

### BRIEF DESCRIPTION OF THE FIGURES

FIG. 01: Illustrates the x-ray powder diffractogram (XRPD) of crystalline form-I of Edoxaban tosylate monohydrate.

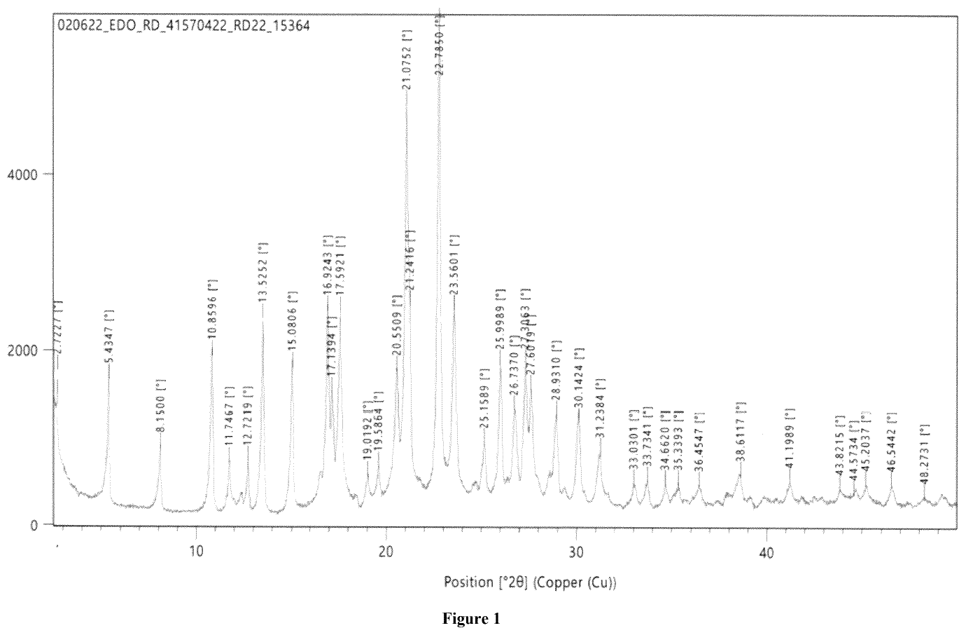

FIG. 02: Illustrates the differential scanning calorimetry (DSC) of crystalline form-I of Edoxaban tosylate monohydrate.

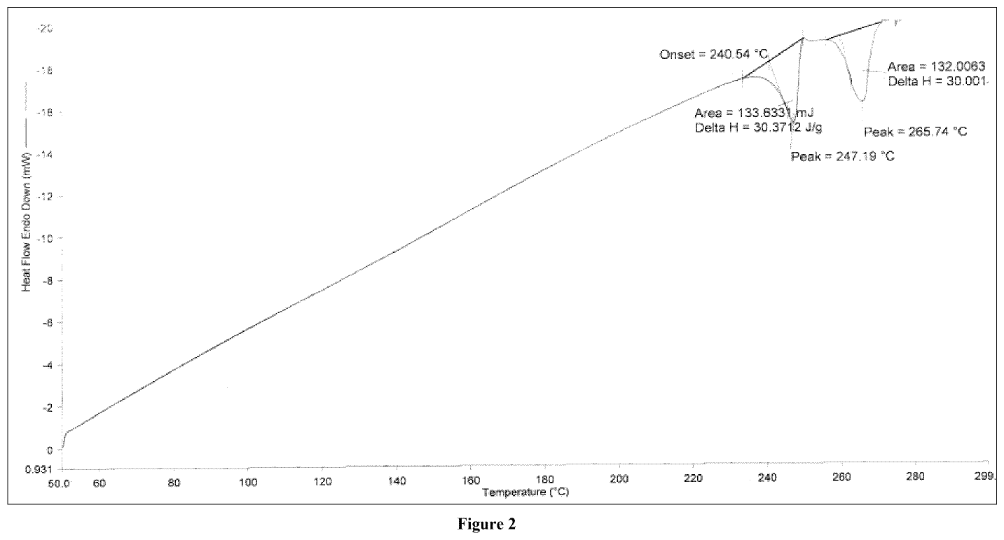

### DEFINITION

All percentages and ratios used herein are by weight of the total composition and all measurements made are at 25° C. and normal pressure unless otherwise designated.

All temperatures used herein are in degree Celsius unless specified otherwise.

All ranges recited herein include the endpoints, including those that recite a range “between” two values.

As used herein, “comprising” means the elements recited, or their equivalents in structure or function, plus any other element or elements that may or may not be recited.

The terms “having” and “including” are also to be construed as open ended unless the context suggests otherwise.

The term “about”, as used herein, refers to any value which lies within the range defined by a number up to +10% of the value.

### DETAILED DESCRIPTION OF THE INVENTION

While the following specification concludes with claims particularly pointing out and distinctly claiming the invention, it is anticipated that the invention can be more readily understood through reading the following detailed description and by studying the included examples.

The best methods and materials of performing the present invention are described here.

The present invention provides a novel, efficient and industrially advantageous process for the preparation of Edoxaban compound of Formula-I or salt thereof.

According to first embodiment, the present invention provides a process for the preparation of Edoxaban of Formula-I or salt thereof,

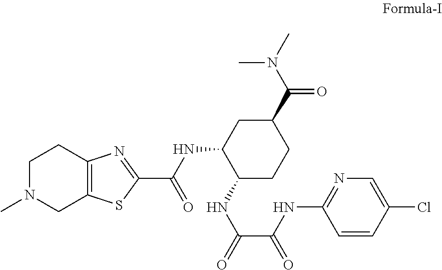 [SMILES: CN1CCc2nc(C(=O)N[C@@H]3C[C@@H](C(=O)N(C)C)CC[C@@H]3NC(=O)C(=O)Nc3ccc(Cl)cn3)sc2C1]

comprising the steps of:
a) reacting compound of Formula-Ia,

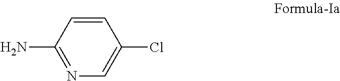

with compound of Formula-Ib,

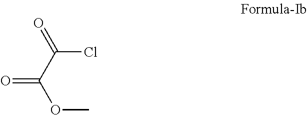

using solvent in absence of base to obtain compound of Formula-II;

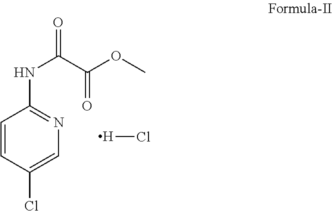

b) reacting compound of Formula-II with compound of Formula-III,

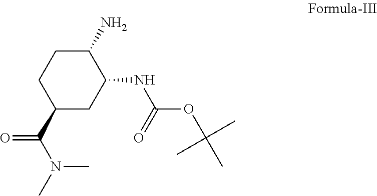 [SMILES: CN(C)C(=O)[C@H]1CC[C@H](N)[C@H](NC(=O)OC(C)(C)C)C1]

in presence of base and solvent to obtain compound of Formula-IV; and

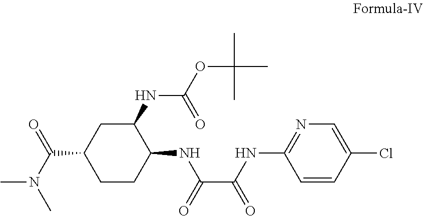 [SMILES: CN(C)C(=O)[C@H]1CC[C@H](NC(=O)C(=O)Nc2ccc(Cl)cn2)[C@H](NC(=O)OC(C)(C)C)C1]

c) converting compound of Formula-IV to Edoxaban of Formula-I or salt thereof.

The compound of Formula-Ia used as a starting material for the preparation of Edoxaban can be prepared by processes known in the art.

In the first embodiment of step a), the solvent is selected from the group consisting of ethyl acetate, isopropyl acetate, n-butyl acetate, or mixture(s) thereof, preferably the solvent is ethyl acetate.

In the first embodiment of step a), the reaction of compound of Formula-Ia with compound of Formula-Ib can be carried out at temperature of about 20° C. to about 70° C., preferably at 60° C. to 65° C.

In the first embodiment of step a), the reaction of compound of Formula-Ia with compound of Formula-Ib can be carried out for 1 hour to 10 hours, preferably for 1 hour to 3 hours.

In the first embodiment of step a), reaction of compound of Formula-Ia with compound of Formula-Ib is carried out in absence of base to obtain compound of Formula-II. After completion of the reaction of compound of Formula-Ia with compound of Formula-Ib resulting mixture can be cooled to temperature of about 20° C. to about 25° C. and filtered to obtained compound of the Formula-II.

The resulting compound of the Formula-II may have purity greater than 99%, preferably greater than 99.3%, more preferably greater than 99.5% by HPLC (High-performance liquid chromatography).

The resulting compound of the Formula-II may have yield greater than 85%, preferably greater than 90%, more preferably greater than 93%.

In the first embodiment of step b), compound of Formula-II can be reacted with compound of Formula-III in presence of base and solvent.

In the first embodiment of step b), the base is selected from the group consisting of organic base or inorganic base.

Inorganic base of step b) is selected from the group consisting of sodium hydroxide, potassium hydroxide, sodium carbonate, sodium bicarbonate or mixture thereof.

Organic base of step b) is selected from group consisting of tertiary amines such as triethylamine, N,N-diisopropylethylamine (DIPEA) or mixture thereof; preferably the base is triethylamine.

In the first embodiment of step b), the solvent is selected from the group consisting of polar aprotic solvent selected from dimethylformamide, dimethyl sulfoxide, N-methylpyrrolidone, acetonitrile or a mixture(s) thereof. Preferably, the solvent is acetonitrile.

In the first embodiment of step b), the reaction of compound of the Formula-II with compound of the Formula-III can be carried out at temperature of about 20° C. to about 70° C., preferably at 55° C. to 60° C.

In the first embodiment of step b), the reaction of compound of Formula-II with compound of Formula-III can be carried out for 1 hour to 10 hours, preferably for 2 hours to 6 hours.

After completion of the reaction of compound of Formula-II with compound of Formula-III, water can be added to the resulting mixture followed by cooling at temperature of about 10° C. to about 20° C. The resulting mixture can be stirred for 30 minutes to 90 minutes. Resulting solid can be filtered and washed with water to obtain wet solid. Resulting wet solid can be dried under vacuum to obtain a compound of the Formula-IV.

The resulting compound of the Formula-IV may have purity greater than 99% by HPLC.

The resulting compound of the Formula-IV may have yield greater than 85%, preferably greater than 90%, more preferably greater 93%.

In the first embodiment of step c), compound of Formula-IV can be converted to Edoxaban of Formula-I or salt thereof, preferably the salt is Edoxaban tosylate monohydrate.

According to second embodiment, the present invention provides a process for preparation of compound of Formula-II,

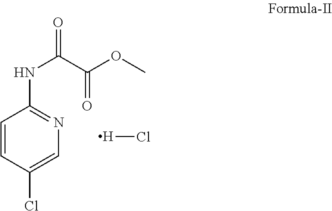

comprising reacting compound of Formula-Ia,

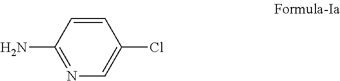

with compound of Formula-Ib,

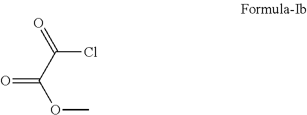

using solvent in absence of base to obtain compound of Formula-II.

In the second embodiment, the solvent is selected from the group consisting of ethyl acetate, isopropyl acetate, n-butyl acetate or mixture thereof, preferably the solvent is ethyl acetate.

In the second embodiment, the reaction of compound of Formula-Ia with compound of Formula-Ib can be carried out at temperature of about 20° C. to about 70° C., preferably at 60° C. to 65° C.

In the second embodiment, the reaction of compound of Formula-Ia with compound of Formula-Ib can be carried out for 1 hour to 10 hours, preferably for 1 hour to 3 hours.

In the second embodiment, the reaction of compound of Formula-Ia with compound of Formula-Ib is carried out in absence of base to obtain compound of Formula-II.

After completion of the reaction of compound of Formula-Ia with compound of Formula-Ib, resulting mixture can be cooled to temperature of about 20° C. to about 25° C. and filtered to obtained compound of the Formula-II.

The resulting compound of the Formula-II may have purity greater than 99%, preferably greater than 99.3%, more preferably greater than 99.5% by HPLC.

The resulting compound of the Formula-II may have yield greater than 85%, preferably greater than 90%, more preferably greater than 93%.

According to third embodiment, the present invention provides a process for preparation of compound of Formula-IV,

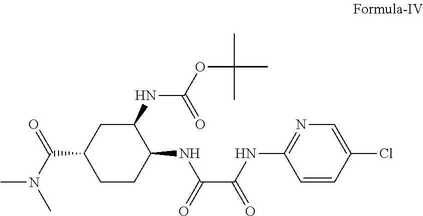 [SMILES: CN(C)C(=O)[C@H]1CC[C@H](NC(=O)C(=O)Nc2ccc(Cl)cn2)[C@H](NC(=O)OC(C)(C)C)C1]

comprising reacting compound of Formula-II,

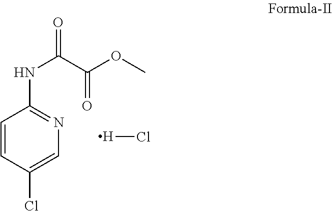

with compound of Formula-III,

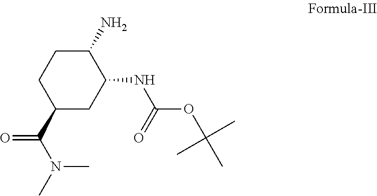 [SMILES: CN(C)C(=O)[C@H]1CC[C@H](N)[C@H](NC(=O)OC(C)(C)C)C1]

in presence of base and solvent to obtain compound of Formula-IV.

In the third embodiment, the base is selected from the group consisting of organic base or inorganic base.

Inorganic base is selected from the group consisting of sodium hydroxide, potassium hydroxide, sodium carbonate, sodium bicarbonate or mixture(s) thereof.

Organic base is selected from group consisting of tertiary amines such as triethylamine, N,N-diisopropylethylamine (DIPEA) or mixture thereof; preferably the base is triethylamine.

In the third embodiment, the solvent is selected from the group consisting of polar aprotic solvent selected from dimethylformamide, dimethyl sulfoxide, N-methylpyrrolidone, acetonitrile or a mixture thereof; preferably the solvent is acetonitrile.

In the third embodiment, the reaction of compound of the Formula-II with compound of the Formula-III can be carried out at temperature of about 20° C. to about 70° C., preferably at 55° C. to 60° C.

In the third embodiment, the reaction of compound of Formula-II with compound of Formula-III can be carried out for 1 hour to 10 hours, preferably for 2 hours to 6 hours. After completion of the reaction of compound of Formula-II with compound of Formula-III, water can be added to the resulting mixture followed by cooling at temperature of about 10° C. to about 20° C. The resulting mixture can be stirred for 30 minutes to 90 minutes. Resulting solid can be filtered and washed with water to obtain a wet solid. Resulting wet solid can be dried under vacuum to obtain a compound of the Formula-IV.

The resulting compound of the Formula-IV may have purity greater than 99% by HPLC.

The resulting compound of the Formula-IV may have yield greater than 85%, preferably greater than 90%, more preferably greater than 93%.

According to fourth embodiment, the present invention provides a process for preparation of Edoxaban compound of Formula-I or salt thereof,

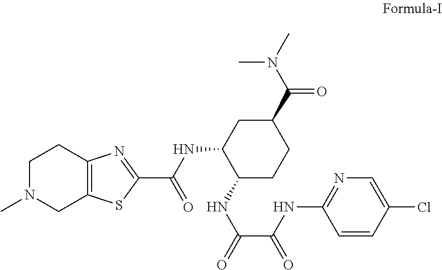 [SMILES: CN1CCc2nc(C(=O)N[C@@H]3C[C@@H](C(=O)N(C)C)CC[C@@H]3NC(=O)C(=O)Nc3ccc(Cl)cn3)sc2C1]

comprising reacting compound of Formula-V,

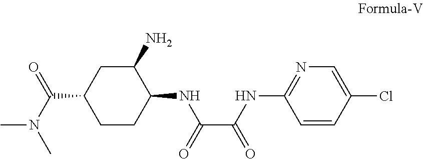 [SMILES: CN(C)C(=O)[C@H]1CC[C@H](NC(=O)C(=O)Nc2ccc(Cl)cn2)[C@H](N)C1]

with compound of Formula-VI,

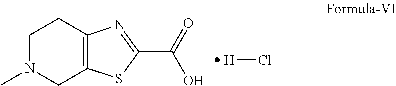

in presence of base, condensing agent, and 2-Hydroxy pyridine-1-oxide (HOPO) as an additive to obtain Edoxaban of Formula-I or salt thereof.

The compound of Formula-V can be prepared by reacting the compound of the Formula-IV with methane sulfonic acid in presence of dichloromethane at temperature of about 20° C. to 25° C. for 2 hours to 3 hours. After completion of reaction, compound of Formula-V can be isolated from the resulting mixture.

Alternatively, resulting mixture containing compound of Formula-V can be taken as such for the next step.

In the fourth embodiment, the amount of 2-hydroxy pyridine-1-oxide (HOPO) used is in the range of 0.09 to 0.19 molar equivalents with respect to compound of the Formula-V.

In the fourth embodiment, the base is selected from the group consisting of organic base or inorganic base; inorganic base is selected from the group consisting of sodium hydroxide, potassium hydroxide, sodium carbonate, sodium bicarbonate or mixture(s) thereof; organic base is selected from group consisting of tertiary amines such as triethylamine, N,N-diisopropylethylamine (DIPEA) or mixture thereof; preferably the base is triethylamine.

In the fourth embodiment, the condensing agent is selected from the group consisting of 1,3-dicyclohexylcarbodiimide (DCC), isobutyl chloroformate, pivaloyl chloride, isovaleryl chloride, 1-ethyl-3-(3-dimethylaminopropyl) carbodiimide hydrochloride (EDC·HCl), 1-cyclohexyl-3-morpholinoethylcarbodiimide, 1-cyclohexyl-3-(4-diethylaminocyclohexyl) carbodiimide, N,N′-carbonyldiimidazole, 2-chloro-1,3-dimethylimidazolinium chloride, isobutyl chloroformate or mixture thereof; preferably the condensing agent is 1-ethyl-3-(3-dimethylaminopropyl) carbodiimide hydrochloride (EDC·HCl).

In the fourth embodiment, the reaction of compound of Formula-V with compound of Formula-VI can be carried at temperature of about 10° C. to about 30° C., preferably at 15° C. to 25° C.

In the fourth embodiment, the reaction of compound of Formula-V with compound of Formula-VI can be carried out for 2 hours to 8 hours, preferably for 5 hours to 8 hours.

Generally, after completion of the reaction of compound of Formula-V with compound of Formula-VI, water can be added to resulting mixture at about 15° C. to about 30° C. pH of the resulting mixture can be adjusted to 7.5 to 8.0 using base and allowed to separate the layers.

Resulting organic layer can be washed with water.

The base can be selected from the group consisting of sodium hydroxide, potassium hydroxide, potassium carbonate, sodium carbonate and sodium bicarbonate; preferably, the base is sodium carbonate.

The obtained organic layer containing Edoxaban can be taken as such for the next step for the preparation of salt, preferably the salt is Edoxaban tosylate or hydrate thereof. Alternatively, the obtained organic layer containing Edoxaban can be partially distilled, followed by addition of isopropanol to obtain a mixture. Resulting mixture can be further distilled to remove maximum dichloromethane and to obtain in situ form Edoxaban in isopropanol. Resulting in situ form Edoxaban in isopropanol solution can be taken for next step for the preparation of salt, preferably the salt is Edoxaban tosylate or hydrate thereof.

Particularly, Edoxaban is reacted with p-toluene sulfonic acid in presence of isopropanol-water to obtain Edoxaban tosylate hydrate. Resulting Edoxaban tosylate hydrate can be recrystallized using isopropanol-water to obtain crystalline form-I of Edoxaban tosylate monohydrate.

The resulting crystalline form-I of Edoxaban tosylate monohydrate may have purity of greater than 99%, preferably greater than 99.2% by HPLC.

Crystalline form-I of Edoxaban tosylate monohydrate encompassed by the present invention may be characterized by at least one of X-Ray powder diffraction (XRPD) and differential scanning calorimetry (DSC). The XRPD pattern of Crystalline form-I of Edoxaban tosylate monohydrate is measured on PANalytical Empyrean diffractometer with copper radiation and expressed in terms of two-theta, d-spacing and relative intensities.

The process for preparation of Edoxaban compound of Formula-I or salt thereof as per the present invention can be summarized in the following schematic representation.

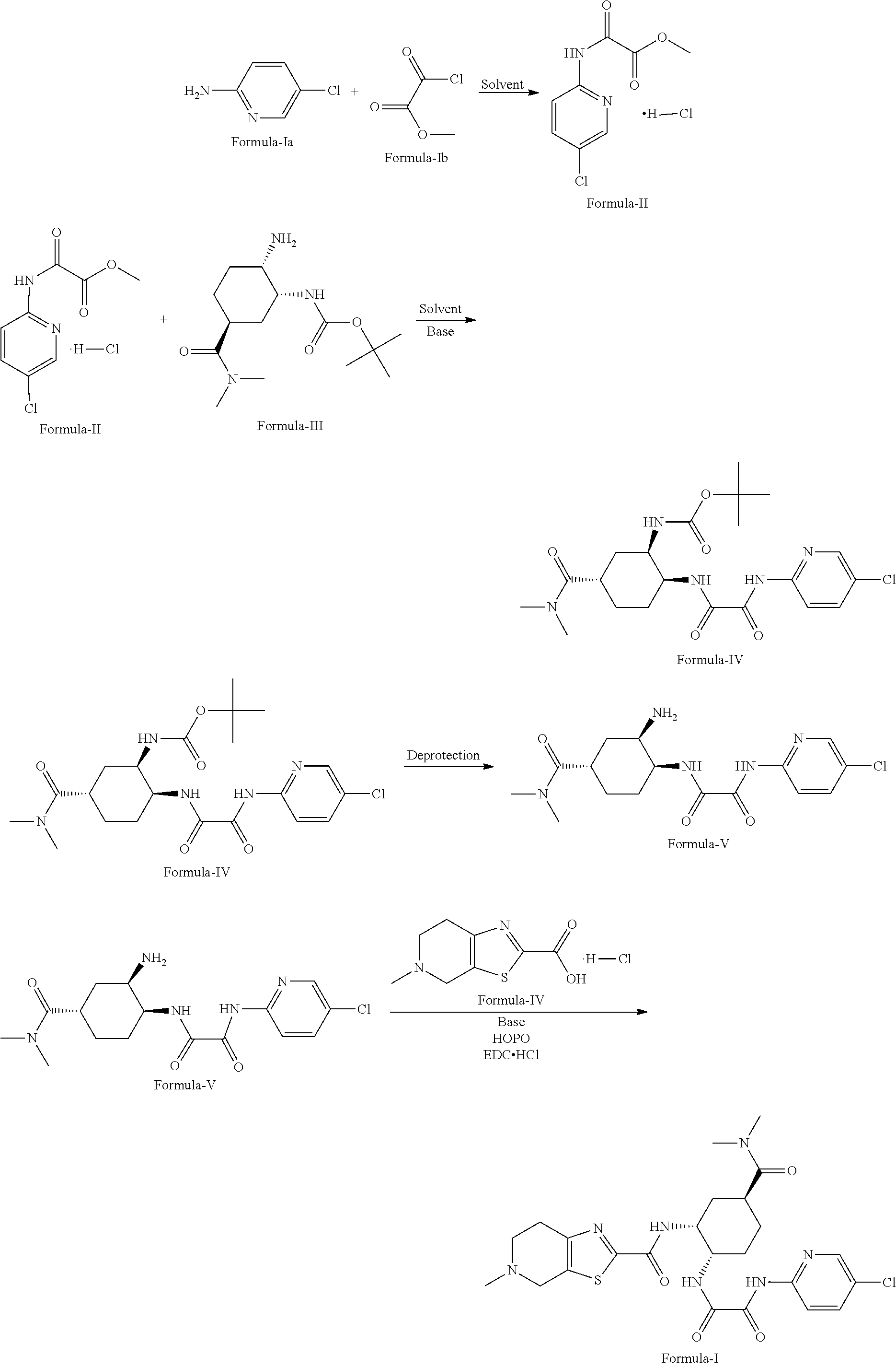

In an attempt to develop an improved process for the preparation of Edoxaban compound of Formula-I or salt thereof and its intermediates to overcome the disadvantages of prior art, the present inventors have developed a process which results in high purity and good yield of Edoxaban compound of Formula-I or salt thereof.

While the present invention has been described in terms of its specific aspects and embodiments, certain modifications and equivalents will be apparent to those skilled in the art and are intended to be included within the scope of the present invention.

### EXAMPLES

The following examples are illustrative of some of the embodiments of the present invention described herein. These examples should not be considered to limit the spirit or scope of the invention in any way.

### Example 01: Preparation of Compound of Formula-II

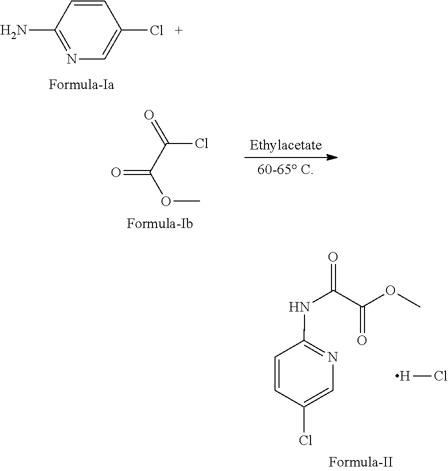

To a stirred solution of ethyl acetate (15.0 L) and 2-amino-5-chloro pyridine (1.0 Kg), methyl oxalyl chloride (1.05 Kg) was added at 60° C. to 65° C. Resulting mixture was stirred for 4 hours. Resulting mixture was then cooled to 20° C. to 25° C. and stirred for 1 hour. The solid obtained was then filtered, washed with ethyl acetate (2.0 L) and dried under vacuum at 60° C. to 65° C. for 7 hours to obtain title compound (1.82 Kg).
Yield: 93.18%
HPLC Purity: 99.60%

### Example 02: Preparation of Compound of Formula-IV

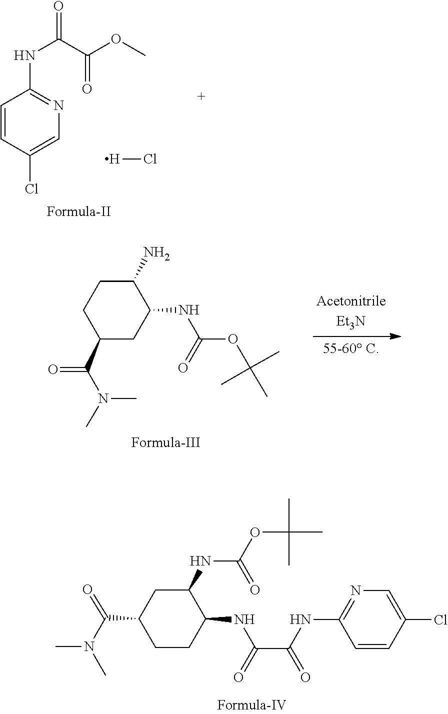

To a stirred solution of acetonitrile (4.0 L) and compound of Formula-III (0.965 Kg), triethylamine (0.60 Kg) was added at 25° C. to 35° C. The resulting mixture was heated at 55° C. to 60° C. Compound of Formula-II (1.0 Kg) obtained in Example 01 was added to the resulting mixture at 50° C. to 60° C. and stirred for 6 hours at same temperature. To the resulting mixture, purified water (4.0 L) was added at 50° C. to 60° C. Resulting mixture was cooled to 10° C. to 20° C. and stirred for 1 hour. The solid obtained was filtered and washed with purified water (1.0 L) at 10° C. to 20° C. Resulting solid was dried under vacuum at 60° C. to 65° C. for 7 hours to obtain title compound (1.5 Kg).
Yield: 94.81%
HPLC Purity: 99.20%

### Example 03: Preparation of Edoxaban Compound of Formula-I

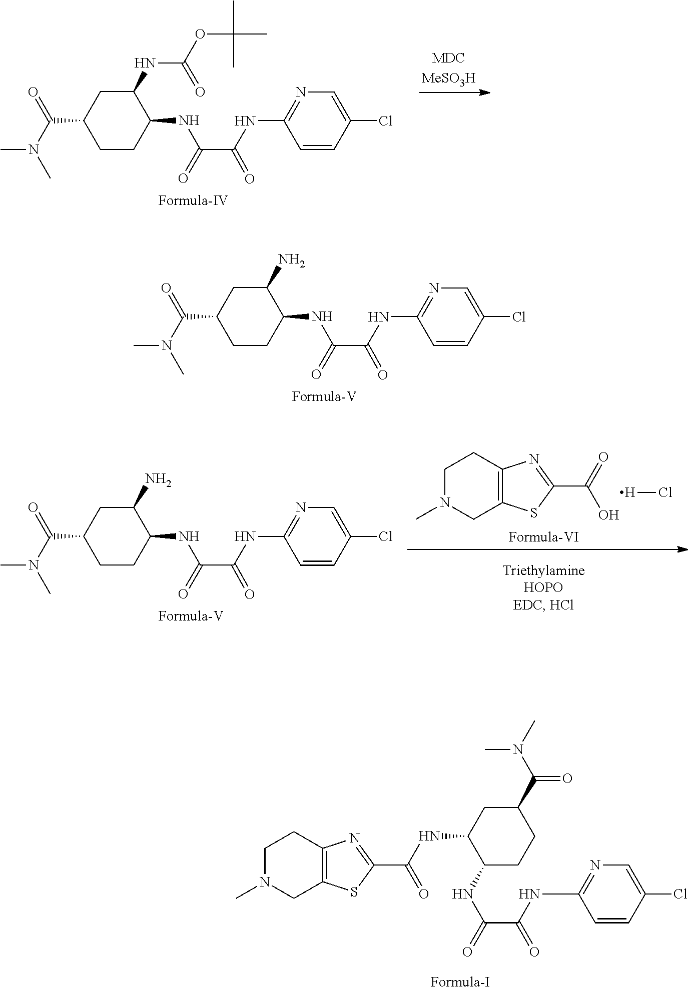

To a stirred solution of dichloromethane (10.0 L) and compound of Formula-IV (1.0 Kg) obtained in Example 02, methane sulphonic acid was added at 20° C. to 25° C. Resulting mixture was stirred for 2 hours at same temperature to obtain mixture containing compound of Formula-V.

Triethyl amine (1.16 Kg) was added to above mixture containing compound of Formula-V at 15° C. to 25° C. To the resulting mixture, compound of Formula-VI (0.55 Kg), 1-(3-dimethylamino-propyl)-ethyl-carbodiimide Hydrochloride (EDC·HCl) (0.45 Kg) and 2-Hydroxy pyridine-1-oxide (HOPO) (0.024 Kg) was added at 15° C. to 25° C. Resulting mixture was stirred at 15° C. to 25° C. for 8 hours. After completion of reaction, purified water (4.0 L) was added to the mixture and stirred at 20° C. to 30° C. for 15 minutes to 20 minutes. pH of the resulting mixture was adjusted to 7.5 to 8.0 using aqueous sodium carbonate at 15° C. to 25° C. and allowed to separate the layers. Resulting layers were separated to obtain organic layer-1 and aqueous layer-1. Resulting organic layer-1 was washed with purified water (4.0 L×3) at 15° C. to 25° C. Resulting organic layer was then distilled under vacuum below 70° C. to obtained solids. Isopropanol was added to the resulting solids and stirred for 20 minutes at 70° C. to 80° C. and further distilled to obtain a title compound having purity of 99.27% by HPLC.

### Example 04: Preparation of Edoxaban Tosylate Monohydrate Compound of Formula-A

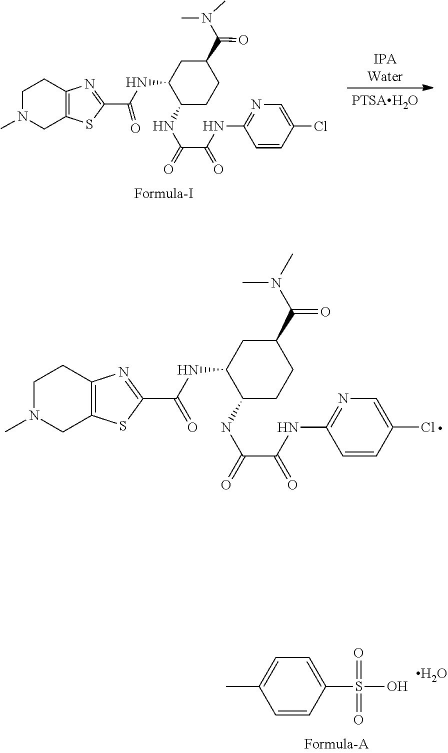

To a stirred solution of isopropanol (10.0 L) and purified water (0.40 L), Edoxaban obtained in Example 03 and p-Toluene sulphonic acid monohydrate (0.45 kg) was added at 25° C. to 30° C. Resulting mixture was heated at 70° C. to 75° C. and stirred for 1 hour. After completion of reaction, resulting mixture was cooled at 10° C. to 20° C. and stirred for 1.0 hour. Resulting solid was filtered, washed with isopropanol (1.0 L) to obtain wet cake. To the resulting wet cake, isopropanol (10.0 L) and purified water (0.40 L) was added at 25° C. to 30° C. resulting mixture was heated at 70° C. to 75° C. and stirred for 1 hour. Resulting solution, was then cooled at 10° C. to 20° C. and stirred for 1.0 hour to precipitate the solid. Precipitated solid was filtered, washed with isopropanol (1.0 L) and dried under vacuum at 60° C. to 65° C. for 6 hours to 8 hours to obtain title compound (1.4 Kg).
Yield: 88.74%
HPLC Purity: 99.32%

### Example 05: Preparation of Edoxaban Tosylate Monohydrate Compound of Formula-A

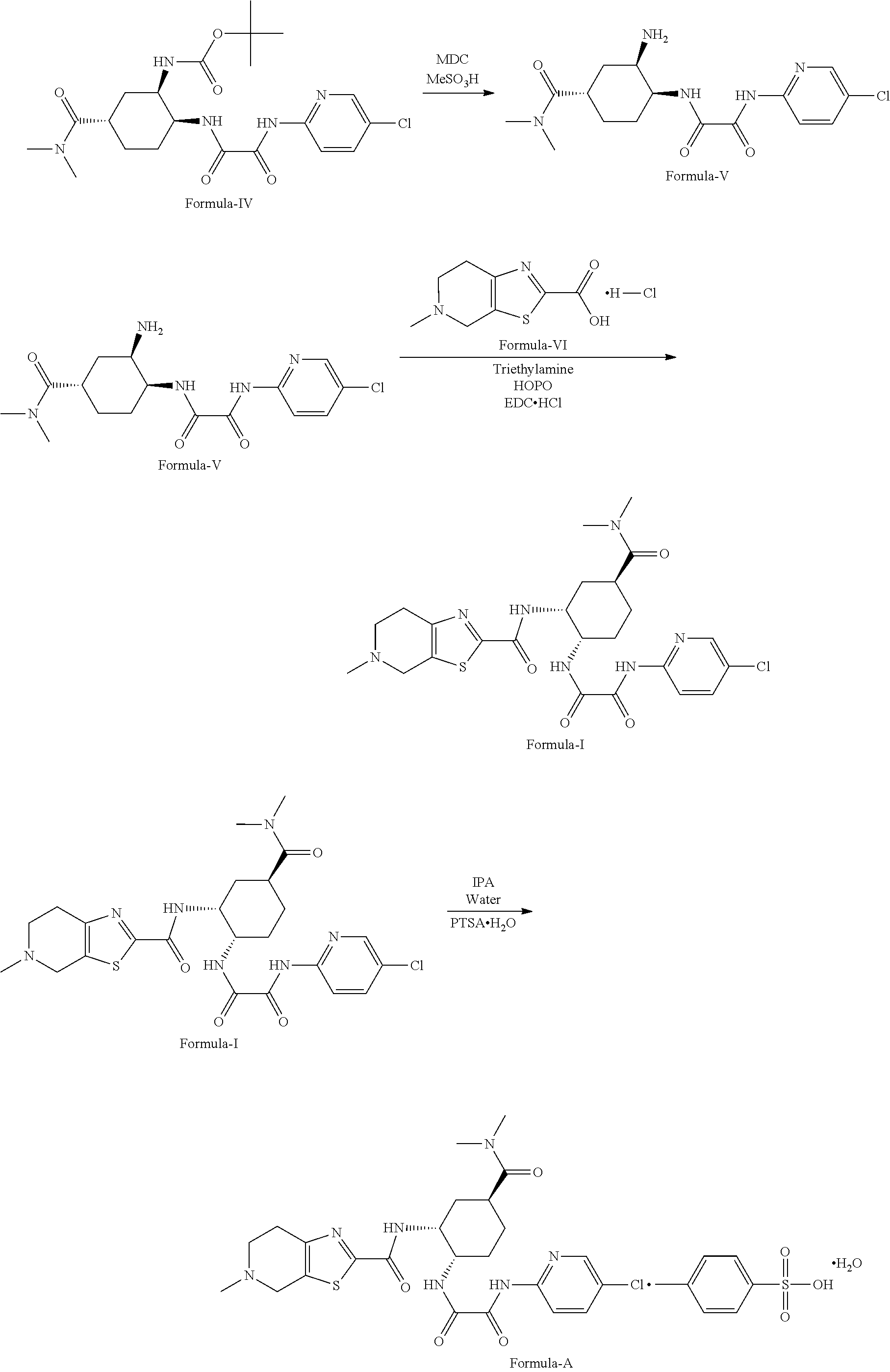

To a stirred solution of dichloromethane (10.0 L) and compound of Formula-IV (1.0 Kg), methane sulphonic acid was added at 20° C. to 25° C. Resulting mixture was stirred for 2 hours at same temperature. Resulting mixture was then cooled at 15° C. to 25° C. to obtain compound of Formula-V.

Triethyl amine (1.16 Kg) was added to above mixture containing compound of Formula-V at 15° C. to 25° C. To the resulting mixture, compound of Formula-VI (0.55 Kg), 1-(3-dimethylamino-propyl)-ethyl-carbodiimide Hydrochloride (EDC·HCl) (0.45 Kg) and 2-Hydroxy pyridine-1-oxide (HOPO) (0.024 Kg) was added at 15° C. to 25° C. Resulting mixture was stirred at 15° C. to 25° C. for 8 hours. After completion of reaction, purified water (4.0 L) was added to the mixture and stirred at 20° C. to 30° C. for 15 minutes to 20 minutes. pH of the resulting mixture was adjusted to 7.5 to 8.0 using aqueous sodium carbonate at 15° C. to 25° C. and allowed to separate the layers. Resulting layers were separated to obtain organic layer-1 and aqueous layer-1. Resulting organic layer-1 was washed with purified water (4.0 L×3) at 15° C. to 25° C. Resulting organic layer was then partially distilled under vacuum below 70° C. followed by addition of isopropanol (10.0 L). Resulting mixture is further distilled to remove dichloromethane.

Purified water (0.40 L) and p-Toluene sulphonic acid monohydrate (0.45 kg) was added to above obtained mixture at 25° C. to 30° C. Resulting mixture was heated at 70° C. to 75° C. and stirred for 1 hour. After completion of reaction, resulting mixture was cooled at 10° C. to 20° C. and stirred for 1.0 hour. Resulting solid was filtered, washed with isopropanol (1.0 L) to obtain wet cake. To the resulting wet cake, isopropanol (10.0 L) and purified water (0.40 L) was added at 25° C. to 30° C. resulting mixture was heated at 70° C. to 75° C. and stirred for 1 hour. Resulting solution, was then cooled at 10° C. to 20° C. and stirred for 1.0 hour to precipitate the solid. Precipitated solid was filtered, washed with isopropanol (1.0 L) and dried under vacuum at 60° C. to 65° C. for 6 hours to 8 hours to obtain title compound (1.4 Kg).
Yield: 88%
HPLC Purity: 99.25%

### Example 06: Purification of Edoxaban Tosylate Monohydrate Compound of Formula-A

To a stirred solution of isopropanol (3.0 L) and purified water (2.0 L), Edoxaban tosylate monohydrate of Formula-A (1.0 Kg) obtained in Example 04 was added at 25° C. to 35° C. Resulting mixture was stirred at 70° C. to 75° C. to obtain clear solution. Activated carbon was added to resulting solution and stirred further for 30 minutes. Resulting hot mixture was filtered on hyflo bed and washed with mixture of isopropanol (0.80 L) and purified water (0.2 L) followed by washing with isopropanol (6.0 L). Resulting filtrate was cooled slowly to 25° C. to 30° C., and further cooled to 0° C. to 5° C. and stirred for 2 hours at same temperature. Resulting solid was filtered, washed with isopropanol (1.0 L) and dried under vacuum at 50° C. to 55° C. for 8 hours to obtain crystalline form-I of Edoxaban tosylate monohydrate (0.88 Kg).
Yield: 88.0%
HPLC Purity: 99.80%

We claim:

1. A process for preparation of Edoxaban of Formula-I or salt thereof 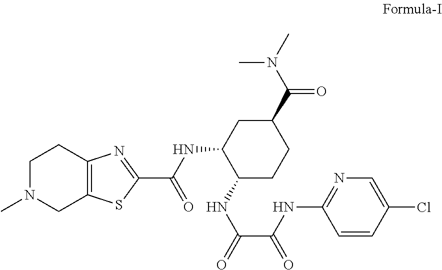 [SMILES: CN1CCc2nc(C(=O)N[C@@H]3C[C@@H](C(=O)N(C)C)CC[C@@H]3NC(=O)C(=O)Nc3ccc(Cl)cn3)sc2C1]
comprising the steps of:
a) reacting compound of Formula-Ia, 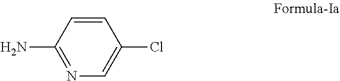
with compound of Formula-Ib, 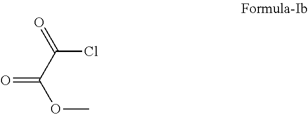
using solvent in absence of base to obtain compound of Formula-II; 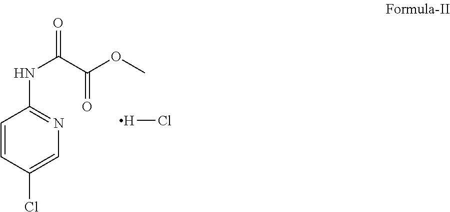
b) reacting compound of Formula-II with compound of Formula-III, 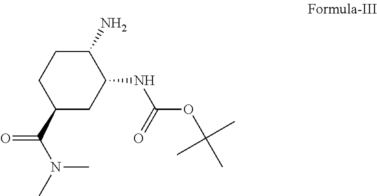 [SMILES: CN(C)C(=O)[C@H]1CC[C@H](N)[C@H](NC(=O)OC(C)(C)C)C1]
in presence of base and solvent to obtain compound of Formula-IV; and 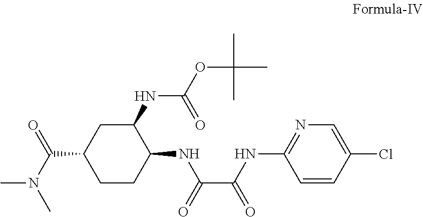 [SMILES: CN(C)C(=O)[C@H]1CC[C@H](NC(=O)C(=O)Nc2ccc(Cl)cn2)[C@H](NC(=O)OC(C)(C)C)C1]
c) converting compound of Formula-IV to Edoxaban of Formula-I or salt thereof.

2. The process as claimed in claim 1, wherein solvent used in step a) is selected from the group consisting of ethyl acetate, isopropyl acetate, n-butyl acetate or mixture(s) thereof.

3. The process as claimed in claim 1, wherein reaction of step a) is carried out at a temperature in the range of 20° C. to 70° C. for 1 hour to 10 hours.

4. The process as claimed in claim 1, wherein solvent used in step b) is selected from the group consisting of polar aprotic solvent selected from dimethylformamide, dimethyl sulfoxide, N-methylpyrrolidone, acetonitrile or mixture(s) thereof.

5. The process as claimed in claim 1, wherein base used in step b) is selected from the group consisting of tertiary amines such as triethylamine and N,N-diisopropylethylamine (DIPEA) or mixture thereof.

6. The process as claimed in claim 1, wherein reaction of step b) carried out at temperature in the range of 20° C. to 70° C. for 2 hours to 6 hours.

7. A process for preparation of compound of Formula-II, 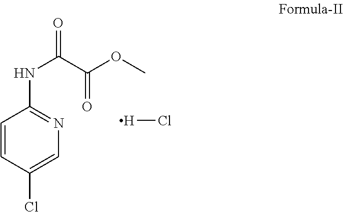
comprising reacting compound of Formula-Ia, 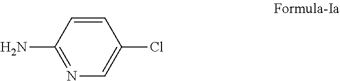
with compound of Formula-Ib, 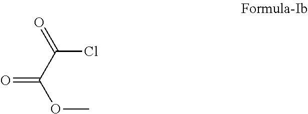
using solvent in absence of base to obtain compound of Formula-II.

8. The process as claimed in claim 7, wherein solvent is selected from the group consisting of ethyl acetate, isopropyl acetate, n-butyl acetate or mixture(s) thereof.

9. The process as claimed in claim 7, wherein reaction is carried out at a temperature in the range of 20° C. to 70° C. for 1 hour to 10 hours.

10. A process for preparation of compound of Formula-IV, 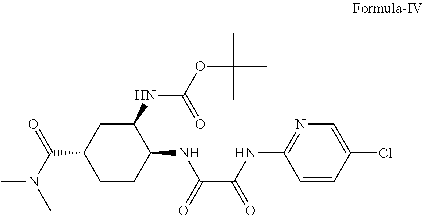 [SMILES: CN(C)C(=O)[C@H]1CC[C@H](NC(=O)C(=O)Nc2ccc(Cl)cn2)[C@H](NC(=O)OC(C)(C)C)C1]
comprising reacting compound of Formula-II, 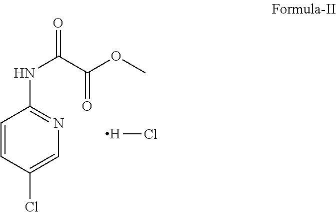
with compound of Formula-III, 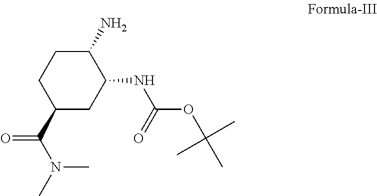 [SMILES: CN(C)C(=O)[C@H]1CC[C@H](N)[C@H](NC(=O)OC(C)(C)C)C1]
in presence of base and solvent to obtain a compound of Formula-IV.

11. The process as claimed in claim 10, wherein solvent is selected from the group consisting of polar aprotic solvent selected from dimethylformamide, dimethyl sulfoxide, N-methylpyrrolidone, acetonitrile or mixture(s) thereof.

12. The process as claimed in claim 10, wherein base is selected from group consisting of tertiary amines such as triethylamine, N,N-diisopropylethylamine (DIPEA) or mixture thereof.

13. The process as claimed in claim 10, wherein reaction is carried out at temperature in the range of 20° C. to 70° C. for 1 hour to 10 hours.

14. A process for preparation of Edoxaban compound of Formula-I or salt thereof, 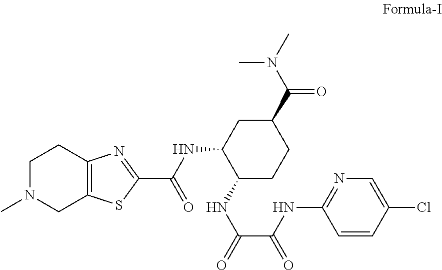 [SMILES: CN1CCc2nc(C(=O)N[C@@H]3C[C@@H](C(=O)N(C)C)CC[C@@H]3NC(=O)C(=O)Nc3ccc(Cl)cn3)sc2C1]
comprising reacting compound of Formula-V, 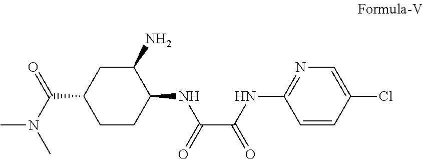 [SMILES: CN(C)C(=O)[C@H]1CC[C@H](NC(=O)C(=O)Nc2ccc(Cl)cn2)[C@H](N)C1]
with compound of Formula-VI, 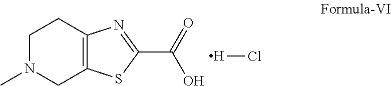
in presence of base, condensing agent, and 2-Hydroxy pyridine-1-oxide (HOPO) as an additive to obtain Edoxaban compound of Formula-I or salt thereof.

15. The process as claimed in claim 14, wherein base is selected from group consisting of tertiary amines such as triethylamine, N,N-diisopropylethylamine (DIPEA) or mixture thereof.

16. The process as claimed in claim 14, wherein condensing agent is selected from group consisting of 1,3-dicyclohexylcarbodiimide (DCC), isobutyl chloroformate, pivaloyl chloride, isovaleryl chloride, 1-ethyl-3-(3-dimethylaminopropyl) carbodiimide hydrochloride (EDC·HCl), 1-cyclohexyl-3-morpholinoethylcarbodiimide, 1-cyclohexyl-3-(4-diethylaminocyclohexyl) carbodiimide, N,N′-carbonyldiimidazole, 2-chloro-1,3-dimethylimidazolinium chloride, isobutyl chloroformate or mixture(s) thereof.

17. The process as claimed in claim 14, wherein reaction is carried out at temperature in the range of 10° C. to 30° C. for 2 hours to 8 hours.
